(19) United States
(12) Patent Application Publication
(10) Pub. No.: US 2014/0100292 A1
(43) Pub. Date: Apr. 10, 2014

(54) Use of moisture-conditioned disintegrants or expanding agents in tablet manufacture for the selective adjustment of the mechanical properties, the dissolving kinetics and/or the water loading of the tablets

(71) Applicants: Eduard BALTHES, Ingelheim am Rhein (DE); Johannes GESER, Gerlingen (DE); Peter SCHNEIDER, Ulm-Einsingen (DE)

(72) Inventors: Eduard BALTHES, Ingelheim am Rhein (DE); Johannes GESER, Gerlingen (DE); Peter SCHNEIDER, Ulm-Einsingen (DE)

(73) Assignee: BOEHRINGER INGELHEIM INTERNATIONAL GMBH, Ingelheim am Rhein (DE)

(21) Appl. No.: 14/048,439

(22) Filed: Oct. 8, 2013

(30) Foreign Application Priority Data
Oct. 9, 2012 (EP) 12187792.2

(51) Int. Cl. A61K 47/38; A61K 47/32; A61K 47/36; A61K 47/34

(52) U.S. Cl. CPC A61K 47/38; A61K 47/34; A61K 47/32; A61K 47/36
USPC 514/772.7; 536/95; 514/772.3; 514/781; 536/102; 514/778; 525/540; 525/326.7

(57) ABSTRACT

The present invention relates inter alia to the use of moisture-conditioned disintegrants or expanding agents in tablet manufacture for the selective adjustment of the mechanical properties, the dissolution kinetics (dissolution) and/or the water loading of tablets.

### FIELD OF INVENTION

The present invention relates inter alia to the use of moisture-conditioned disintegrants or expanding agents in tablet manufacture for the selective adjustment of the mechanical properties, the dissolving kinetics (dissolution) and/or the water loading or moisture content of tablets.

### BACKGROUND OF THE INVENTION

### Definitions

The terms “expanding agents” or “disintegrants” are hereinafter combined under the term “disintegrants”. The tablets as mentioned herein may relate, in one embodiment, to tablet cores or, in another embodiment, to film-coated tablets.

Critical humidity/critical humidity range: the humidity level at which the disintegrant swells so rapidly or to such a volume as a result of water uptake that the resulting swelling force enables the interparticulate forces or interactions to be overcome, and as a result gives rise to mechanical instability or fracturing in the tablet.

Selectively moisture-conditioned disintegrant: disintegrant that has been conditioned under specific climatic conditions (humidity/temperature) and has thus been pre-swollen to a defined extent by water uptake but still has sufficient residual disintegration force to cause the tablet to decompose when the product is wetted with aqueous liquid. The details of the conditioning have to be determined experimentally dependent on the product properties (composition/process parameters).

Unconditioned disintegrant: disintegrant that has not been adjusted to a defined moisture content. The actual moisture content is generally not known or not explicitly determined before the processing.

Physical stability: the tablet is mechanically intact and shows no damage caused by swelling of the disintegrant as a result of water absorption, such as fractures, for example. This takes no account of chemical stability.

Put in very simple terms, the tablets are produced by initially mixing generally powdered or granular active substances of a defined dosage with powdered or granular adjuvants in an defined amount to form a homogeneous mass. This mixture is then compressed into tablets under defined conditions (such as e.g. temperature, relative humidity, punch pressure applied, pressure-time profile of the pressing, etc.). The hardness of the tablets obtained is critically determined by the statistical distribution of the particle sizes of the components, the degree of mixing of the various components, the material, morphology-specific and particle-size-dependent interactive forces of the adjacent particles of the formulation and the manufacturing parameters mentioned above. This hardness is one of the determining factors for the physical stability of a tablet under mechanical, thermal and/or humidity loading as a function of time. Moreover, the hardness inter alia also determines the disintegration and hence the dissolution of a tablet after it is taken and accordingly the release kinetics of the active substance. A specific required hardness on the one hand can therefore shift the dissolution kinetics of a tablet into a very unfavourable range on the other hand or even limit them to a narrow window. To compensate for this, disintegrants are often added to the formulation. By their moisture-dependent expansion of volume (swelling) they ensure that the tablet disintegrates after being taken by the patient. The disintegration (of the tablet core or the film-coated tablet) abruptly increases the effective surface area and thus speeds up the dissolution of the tablet and/or the release of the active substance.

Unfortunately, at present, the level of critical humidity at which a tablet disintegrates can only be influenced very slightly, if at all, by the formulation or tabletting. Certainly it is possible to choose from different disintegrants, but the number of useable disintegrants actually remaining under the marginal conditions of for example compatibility with the formulation, physical/chemical properties and workability is usually very limited. Variation in the granule size, for example, is only of limited value with regard to its effect on the tabletting. The use of unconditioned disintegrant may therefore have the effect of causing a tablet to break into fragments prematurely, i.e. before it is taken, or to disintegrate (such as e.g. in its granulate- or active substance/adjuvant-particles) partially or completely, i.e. shatter. This may happen during storage within the shelf-life or in very unfavourable cases may even occur during manufacture or may critically reduce the storage shelf-life as prior damage during manufacture. Typically, disintegration of tablets is only desired in in vitro dissolution/disintegration experiments or in in vivo administration by the patients, otherwise, the tablets should be mechanically stable enough to allow production, transport and storage under typical storage conditions in the pharmaceutical industry, in commerce and at patients within the required storage conditions and terms.

The disintegration of a tablet takes place when the moisture-dependent volume expansion of the disintegrant in the tablet leads locally to disintegrating forces which are greater than the forces acting between the granular or powdered components of the formulation. These are determined inter alia by the process conditions during tabletting and by the demands made of the mechanical stability (e.g. hardness, abrasion) of the tablet. The disintegration forces in turn are determined by the moisture-dependent expansion in volume of the disintegrant used (however, other disintegration accelerators or disintegrants are known that may bring about breakup or disintegration by another mechanism, e.g. a wick effect). The interplay between these two forces therefore determines the critical humidity range at which a tablet disintegrates relatively precisely (but hardly in a controllable manner. As already mentioned, this humidity range is often within the operating range (with respect to relative humidity, r.h.) of the manufacturing process itself, so that even during manufacture preliminary damage to the product often cannot be ruled out. To prevent this some complex counter-measures are required (e.g. after-drying, conditioning of tablets, short holding times during production, storage and handling of the bulk goods and packaging) and at the same time expensive packaging often has to be developed to keep the product in a specific humidity range throughout its shelf-life and during the in-use time once the packet has been opened. Generally, the effect of the moisture-induced preliminary damage is also additionally temperature-dependent, so that in the delivery chain after manufacture greater efforts have to be made to avoid damage to the product. As a result, the use of unconditioned disintegrants may sharply reduce the moisture and temperature range for the safe manufacture and storage of tablets.

### SUMMARY OF THE INVENTION

Within the scope of the present invention the use of one or more selectively moisture-conditioned disintegrants in the respective formulations is proposed, in order to avoid an adverse effect on the physical stability or storage qualities of the formulation present, caused by uncontrolled, premature and/or excessive absorption of moisture or swelling of the disintegrant, or to obtain corresponding optimisation with regard to physical stability or storage qualities.

It is also proposed within the scope of the present invention, in the manufacture of tablets which have hitherto contained an unconditioned disintegrant in the formulation and the physical stability or storage qualities of which are adversely affected by the swelling of the disintegrant as a result of water absorption, e.g. during production, packaging or storage as described above, to replace the unconditioned disintegrant in the formulation by a selectively moisture-conditioned disintegrant, if the chemical stability of the product allows.

This is intended to keep the tablets physically stable even at a higher relative humidity than the one at which they usually show mechanical damage when the disintegrant is processed in the dry state with maximum disintegrating force.

In addition the use of a moisture-conditioned disintegrant is intended to widen the relative humidity and temperature range of production and/or storage of tablets, to simplify their manufacture, minimise the costs of suitable packaging for them and/or at the same time increase their shelf-life.

### BRIEF DESCRIPTION OF THE DRAWINGS

FIG. 1 shows the schematic representation of the equilibrium humidity (scale on the left) and equilibrium water content (scale on the right) at thermal equilibrium before ((a), (b), (c), (d)) and after tabletting ((e)) and critical values of the fragmentation ((f)) and climatic conditions ((d)) during storage, in Cases 1-4 (cf. Examples 2a-2d as described herein) using different conditioned components. All the equilibrium humidity r.h. values have been chosen randomly for illustrative purposes: (c) denotes the r.h. of the active substance (including any other adjuvants with the exception of the disintegrant), (d) denotes the r.h. of the disintegrant, (e) denotes the resulting equilibrium r.h. of the finished tablet.

### DETAILED DESCRIPTION OF THE INVENTION

Accordingly, the present invention provides a formulation (particularly a solid pharmaceutical formulation or composition, for example in the form of a tablet), which contains as disintegrant a selectively moisture-conditioned disintegrant.

Moreover, the present invention relates to a formulation (particularly a solid pharmaceutical formulation, blend, preparation or composition, for example in the form of a tablet) comprising (or essentially consisting of) a disintegrant, optionally together with one or more active substances and/or other adjuvants, the disintegrant being a selectively moisture-conditioned disintegrant.

Moreover, the present invention relates to a formulation (particularly a solid pharmaceutical formulation or composition, for example in the form of a tablet) comprising or essentially consisting of:
one or more active substances,
one or more selectively moisture-conditioned disintegrants,
and optionally one or more other adjuvants.

The invention further proposes the use of at least one selectively moisture-conditioned disintegrant in a formulation (particularly a solid pharmaceutical formulation or composition, for example in the form of a tablet).

The invention further provides a method for preventing or reducing the (uncontrolled, premature and/or excessive) swelling of a disintegrant by water absorption in a formulation (particularly a solid pharmaceutical formulation or composition, for example in the form of a tablet), the method comprising the use of a selectively moisture-conditioned disintegrant as disintegrant within the formulation.

The invention further provides a method for improving the hardness, the physical stability, the shelf-life and/or the storage qualities of a disintegrant-containing formulation (particularly a solid pharmaceutical formulation or composition, for example in the form of a tablet), the method comprising the use of a selectively moisture-conditioned disintegrant as disintegrant within the formulation.

The present invention further relates to the use of a selectively moisture-conditioned disintegrant within a formulation (particularly a solid pharmaceutical formulation or composition, for example in the form of a tablet) for improving the hardness, the physical stability, the shelf-life and/or the storage qualities of the formulation.

Moreover, the present invention relates to the use of a selectively moisture-conditioned disintegrant, and optionally one or more active substances and/or other adjuvants, for preparing a formulation (particularly a solid pharmaceutical formulation or composition, for example in the form of a tablet) with improved hardness, physical stability, shelf-life and/or storage qualities.

Moreover, the present invention relates to a method for preparing a formulation (particularly a solid pharmaceutical formulation or composition, for example in the form of a tablet), comprising the use of a selectively moisture-conditioned disintegrant.

Moreover, the present invention relates to a method for preparing a formulation (particularly a solid pharmaceutical formulation or composition, for example in the form of a tablet), comprising mixing a disintegrant with one or more active substances and/or one or more other adjuvants, the disintegrant being a selectively moisture-conditioned disintegrant.

Moisture-conditioned components (particularly disintegrants) may be produced for example by open storage or by storing in moisture-permeable packaging in a specific climate (defined temperature and relative humidity). Alternatively these moisture-conditioned materials may also be produced for example by mixing highly moisture-laden or saturated material (disintegrant) with dry material (disintegrant). From the sorption capacity of the material (disintegrant) depending on the relative humidity and temperature provided, the mixing ratio of saturated and dry material (disintegrant) can be calculated which yields the desired relative equilibrium humidity of the mixture at a specific temperature, i.e. the moisture-conditioning of the material (disintegrant).

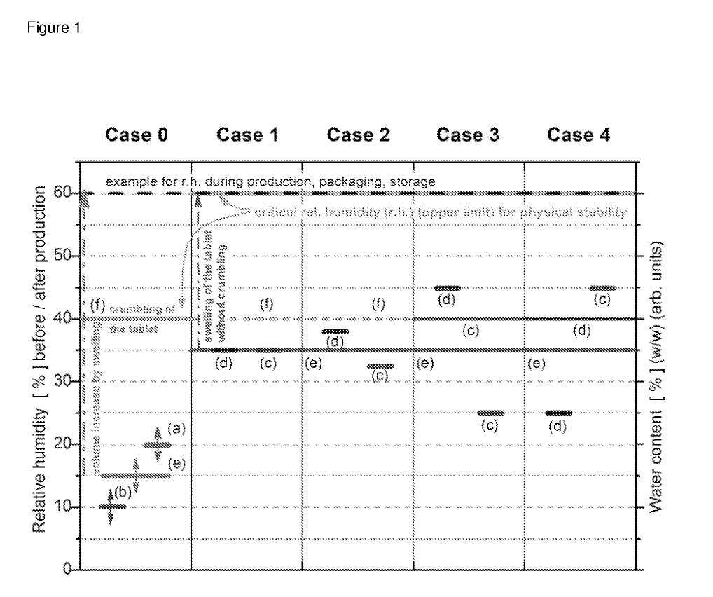

The sorption capacity (water loading) of disintegrants is a continuous function dependent on the relative humidity (and the temperature). Accordingly the swelling (expansion in volume) of a disintegrant as a specific material property is a continuous function that crucially co-determines resulting disintegration forces in a formulation. The use of moisture-conditioned disintegrant leads to the particles thereof being already pre-swollen to a specifically preset degree in the processing state. The effect that can be achieved may be characterised as follows, for example (cf. Examples 1, 2a-2d, and FIG. 1):

The following Examples 2a-2d describe the advantages and the adjustment possibilities achieved for the physical or physical-chemical properties of tablets produced from moisture-conditioned components, particularly using moisture-conditioned disintegrants. By comparison, Example 1 describes the properties of tablets produced from unconditioned components.

### EXAMPLE 1

Properties of the formulation components before tabletting: all the components of the tablet, i.e. disintegrant and active substance (including any other adjuvants) are unconditioned.

A tablet “T1” is prepared with an unconditioned, i.e. dry, non-preswollen disintegrant+active substance and in a climate of e.g. 25° C./60% r.h.

T1 absorbs a relatively large amount of water, and disintegrates prematurely as a result of the excessive swelling of the disintegrant, i.e. at too low a relative humidity or too rapidly in the range of a critical r.h. The disadvantages of this conventional procedure are the often premature fragmentation of the tablet (i.e. damage even at low relative humidity levels, critical limit here e.g. 40% r.h.) and/or fluctuations in the quality of the tablet in terms of its mechanical stability, e.g. due to variations in the initial humidity and therefore variations in the absorption of moisture (=swell reserve) up to the point of disintegration.

### EXAMPLE 2

By comparison, a tablet “T2” is produced with a disintegrant preconditioned to a specific relative humidity. As a result of the preswelling of the disintegrant caused by its moisture conditioning the additional swelling during storage (in this case e.g. 60% r.h./25° C.) is less than in T1 and thus the disintegration forces in T2 are also less than in T1. The disintegration thus only occurs at a higher relative humidity, compared with Example 1, and possibly only after a time delay. In addition, the relative humidity beyond which the disintegration of or damage to the formulation takes place can be (selectively) adjusted by the choice of the conditioning humidity of the components (active substance and/or disintegrant), and hence by their swell volume. Moreover, in this way, the delay in disintegration can be selectively adjusted. Thus, this method can also be used to produce delayed-release pharmaceutical preparations. This and the possibilities arising from it for improved tablet production are given as Examples 2a-2d:

### EXAMPLE 2a

Properties of the formulation components before tabletting: all the components of the tablet, i.e. disintegrant and active substance (including any other adjuvants) are conditioned to the same specific relative humidity, and both conditioning processes are below a specific critical limit (in this case e.g. 40% r.h.).

One advantage of this procedure according to the invention is:
i) during production, packaging and/or storage under specific climate conditions (in this case e.g. 60% r.h.), the disintegrants is preswollen during tabletting, i.e. a higher humidity is needed to develop the disintegrating force. Therefore the tablet remains mechanically stable up to a high humidity.

Other advantages of this procedure according to the invention are:
ii) By the choice of the relative humidity of the conditioning of the components, particularly of the disintegrant, matched to other influencing factors in the tabletting (tablet punch, pressure-time profile) the time at which disintegration occurs can be adjusted selectively.
iii) It is possible to adapt the physical properties (e.g. hardness, release kinetics of the active substance) to the claims of the product during production and storage by the choice of the particular disintegrant: the amount of water introduced (determined by the sorption capacity of the disintegrant) can be matched to the release kinetics of the active substance and to the sensitivity of the product and the target equilibrium humidity during processing and/or storage.
iv) It is possible to match disintegration forces (hardness) to the formulation or to the binding forces of adjuvants.

### EXAMPLE 2b

Properties of the formulation components before tabletting: disintegrant or active substance (including any other adjuvants) are conditioned to a specific, different relative humidity, and both conditioning processes are below a certain critical limit (e.g. 40% r.h.). Advantages of this procedure according to the invention are: see under Example 2a i) to iv).

Other advantages of this procedure according to the invention are:
v) As the disintegrant is conditioned at a higher humidity before tabletting than that which corresponds to the state of equilibrium after tabletting, a “free volume” is formed after the tabletting, or a relaxation of the mixture in the immediate vicinity of the granulated disintegrant. This allows a selectively adjustable delayed disintegration (sustained release effect) which can be utilised for example:
v1) either to shift the “disintegration limit”, i.e. the critical relative humidity at which disintegration begins, upwards (as there is more swell reserve/space around the disintegrant), v2) or to give the tablet greater hardness in this way (disintegrant “does not press on the tablet structure”),
v3) or as a possible way to lower the equilibrium humidity for the finished tablet if necessary (e.g. if the starting components of the formulation do not have a common optimum humidity range for their handling/processing).
v4) Options v1) and v3) also include the possibility of matching the moisture that is to be absorbed between the optimum storage humidity and disintegration as a function of the moisture sensitivity of the product and the desired hardness of the tablet.

### EXAMPLE 2c

Properties of the formulation components before tabletting: disintegrant and/or active substance (including any other adjuvants) are conditioned to a specific different relative humidity, and conditioning of the disintegrant is above a specific critical limit (e.g. 40% r.h.).

Advantages of this procedure according to the invention are: see under Example 2a i) to iv), and 2b v).

Other advantages of this procedure according to the invention are:
vi) The r.h. of the disintegrant may be above a r.h. that is critical for the active substance, at which breakdown of the active substance sets in. Nevertheless, a specific lower equilibrium humidity below the above-mentioned critical rel. humidity can be adjusted in the finished tablet. This option is advisable if the active substance has to be processed and stored below a certain moisture limit.
vii) This can be achieved by not mixing the (optionally conditioned) active substance and the conditioned disintegrant until shortly before the tabletting.

### EXAMPLE 2d

Properties of the formulation components before tabletting: disintegrant and/or active substance (including any other adjuvants) are conditioned to a specific different relative humidity, and conditioning of the active substance is above a specific critical limit (e.g. 40% r.h.).

Advantages of this procedure according to the invention are: see under Example 2a i) to iv).

Other advantages of this procedure according to the invention are:
viii) This method is advantageous if the disintegrant cannot be stored or processed in too much humidity, e.g. owing to clumping, blockage of transfer devices, etc.
ix) This provides the possibility of drying the active substance “within the tablet” after tabletting, e.g.:
ix1) if the active substance cannot be stored under dry enough conditions (e.g. on account of electrostatic problems, the ability to meter it during the transfer process, dust, etc.),
ix2) if the active substance is produced in aqueous or moist medium and can only be dried to a limited extent before the tabletting,
x) This can be achieved by not mixing the conditioned active substance and the (optionally conditioned) disintegrant until shortly before tabletting.
xi) The preconditioning of the components can be selectively chosen so as to adjust the after-swelling of the disintegrant after tabletting by means of the difference in the equilibrium humidity of the pre-conditioned disintegrant and the finished tablet. The resulting disintegration forces may if necessary be used as “pre-stressing” of the tablet in order to deliberately bring about fragmentation at a lower relative humidity than would be the case without the “pre-stressing”. In this way the relative humidities of storage of the components, tabletting, storage of the tablet and critical humidity of the fragmentation (disintegration) can be deliberately matched to one another (cf. 2b v4)).
xii) This method can also be used to selectively reduce or adjust the hardness of a tablet by means of the adjustable equilibriums described in xi) and the resulting pre-stressing.

Cases 1-4 (cf. relevant Examples 2a-2d) of FIG. 1 show the procedure according to the invention using conditioned components.

By the choice of conditioning of the components illustrated in Examples 2a-2d, but also by the choice of the nature of the disintegrant it is possible to expand the humidity range within which a disintegrant does not break the tablet apart, i.e. within which the tablet remains mechanically stable, or to critically influence the rel. humidity at which it is then disintegrated. When selecting the nature of the disintegrant the moisture input can be used as a deciding criterion and/or the expansion in volume during swelling and the associated disintegration forces. Particular mention should be made of a relatively recently developed disintegrant, a so-called cross-linked polyalkylammonium polymer (e.g. produced by cross-linking of 1,10-dibromodecane and 1,6-diaminohexane, sold e.g. under the trade name “DMP 504”). This material has a relatively low water absorption at rel. humidities of up to 70% and then at higher humidity levels within a narrow humidity range it takes up very much more water than comparable disintegrants. This allows a small incorporation of water (gentle on the active substance) during conditioning to low rel. humidities and promises a disintegrating force just as high as before, at high relative humidities. Other disintegrants that may be considered are e.g. crospovidone and derivates thereof (e.g. with different particle sizes, type A and B), croscarmellose, sodium starch glycolate, starches and derivatives thereof and in principle all known expanding agents and/or disintegrants.

It should be mentioned that these above-mentioned disintegrants have hitherto only been sold and utilised for use as “normal”, i.e. unconditioned disintegrants. According to the present invention they may be used as conditioned disintegrants.

Within the scope of the present invention it is proposed, e.g. in the cases described above, to switch from the processing of unconditioned disintegrants in tablet formulations to the use of moisture-conditioned disintegrants.

By this method as described herein, the precise value of the relative humidity at which the disintegration of a tablet sets in can be adjusted for the first time. This would be useful for critically prolonging the stability of products and greatly simplifying the manufacturing and packaging processes in the manner of a Quality-by-Design process: With the degree of pre-conditioning required, attention can be paid to the product-specific demands and requirements from the manufacturing process right up to the demands made of the mechanical stability of the product. The degree of moisture conditioning required can be determined from the correlation between water absorption, expansion in volume and the resulting disintegration forces, or can be determined experimentally directly on tablets produced in tests with a specific, differently conditioned disintegrant.

Typically, for example, the degree of selectively desired or necessary moisture conditioning of the material may be in the range from 0.1% to 20% w/w water content in the material, such as e.g. from 0.5% to 5% w/w. Preferably, the moisture pre-conditioning of the formulation is in the range from 1.0% to 5% w/w, even more strictly in the range from 1.5% to 4% w/w (moisture content of the formulation).

In the tablet production, a working point with regard to the relative humidity can be deliberately determined and controlled as a function of the properties of the product and its processing. During storage a critical humidity and/or a critical storage period can be selectively varied.

Examples of a product in which the method proposed here could be used may include any tablet formulation in which an expanding agent or disintegrant, or an adjuvant capable of bringing about an unintended disintegration of the tablet, is used. Moisture conditioning of adjuvants in matrix tablets is also possible.

### FURTHER EMBODIMENTS OF THE INVENTION

1. Formulation (particularly a solid pharmaceutical formulation or composition, for example in the form of a tablet), which contains a selectively moisture-conditioned disintegrant as the disintegrant.

2. Formulation (particularly a solid pharmaceutical formulation or composition, for example in the form of a tablet) comprising or essentially consisting of:
one or more active substances,
one or more selectively moisture-conditioned disintegrants,
and optionally one or more other adjuvants.

3. A disintegrant-containing formulation (particularly a solid pharmaceutical formulation or composition, for example in the form of a tablet) e.g. according to embodiment 2, particularly with improved hardness, physical stability, shelf-life and/or storage qualities, wherein a selectively moisture-conditioned disintegrant is present instead of an unconditioned disintegrant.

4. Use of a selectively moisture-conditioned disintegrant, and optionally one or more active substances and/or other adjuvants, for preparing a formulation (particularly a solid pharmaceutical formulation or composition, for example in the form of a tablet) with improved hardness, physical stability, shelf-life and/or storage qualities.

5. Method for preventing or reducing the unwanted (uncontrolled, premature and/or excessive) swelling of a disintegrant in a formulation (particularly a solid pharmaceutical formulation or composition, for example in the form of a tablet), the method comprising the use of a selectively moisture-conditioned disintegrant as disintegrant within the formulation.

6. Method for improving the hardness, the physical stability, the shelf-life and/or the storage qualities of a disintegrant-containing formulation (particularly a solid pharmaceutical formulation or composition, for example in the form of a tablet), the method comprising the use of a selectively moisture-conditioned disintegrant as disintegrant within the formulation.

7. Formulation or use according to embodiment 1, 2, 3 or 4, wherein the disintegrant and the active substance (including any other adjuvants) are each conditioned to the same specific relative humidity, and both conditioning processes are below a specific critical relative humidity limit for fragmentation and/or disintegration of the tablet.

8. Formulation or use according to embodiment 1, 2, 3 or 4, wherein the disintegrant and the active substance (including any other adjuvants) are each conditioned to different specific relative humidities, and both conditioning processes are below a specific critical relative humidity limit for fragmentation and/or disintegration of the tablet.

9. Formulation or use according to embodiment 1, 2, 3 or 4, wherein the disintegrant and the active substance (including any other adjuvants) are each conditioned to different specific relative humidities, and the conditioning of the disintegrant is above a specific critical relative humidity limit for fragmentation and/or disintegration of the tablet, and the conditioning of the active substance (including any other adjuvants) is below a specific critical relative humidity limit for fragmentation and/or disintegration of the tablet.

10. Formulation or use according to embodiment 1, 2, 3 or 4, wherein the disintegrant and the active substance (including any other adjuvants) are each conditioned to different specific relative humidities, and the conditioning of the disintegrant is below a specific critical relative humidity limit for fragmentation and/or disintegration of the tablet, and the conditioning of the active substance (including any other adjuvants) is above a specific critical relative humidity limit for fragmentation and/or disintegration of the tablet.

11. Method for preparing a formulation (particularly a solid pharmaceutical formulation or composition, for example in the form of a tablet), comprising mixing or combining a disintegrant with one or more active substances and/or one or more other adjuvants, the disintegrant being a selectively moisture-conditioned disintegrant.

12. Formulation, use or method according to at least one of embodiments 1 to 11, wherein the moisture-conditioned disintegrant can be prepared:
by open storage or storage of the disintegrant in a moisture-permeable packaging under specific climatic conditions, or
by mixing highly moisture-laden moisture-saturated disintegrants with dry disintegrants, in order to achieve the selective moisture conditioning of the disintegrant in this way.

13. Formulation, use or method according to at least one of embodiments 1 to 12, wherein the selectively moisture-conditioned disintegrant is a selectively moisture-conditioned cross-linked polyalkylammonium polymer (e.g. DMP 504), crospovidone (including derivates thereof having different particle sizes, type A or B), croscarmellose, starches or derivatives thereof, or sodium starch glycolate.

14. Formulation, use or method according to at least one of embodiments 1 to 13, wherein the selectively moisture-conditioned disintegrant is in a formulation with a water content in the range from 0.1% to 20% w/w, preferably from 0.5% to 5% w/w.

15. Formulation, use or method according to at least one of embodiments 1 to 13, wherein the selectively moisture-conditioned disintegrant is obtained by moisture conditioning to obtain a water content for the formulation in the range from 1.0% to 5% w/w, more strictly within the range from 1.5% to 4% w/w.

16. Formulation, use or method according to at least one of embodiments 1 to 15, wherein the formulation (particularly a solid pharmaceutical formulation or composition, for example in the form of a tablet) has a (specified) delayed disintegration and/or a delaying effect on the release of active substance.

17. Formulation, use or method according to at least one of embodiments 1 to 15, wherein the formulation (particularly a solid pharmaceutical formulation or composition, for example in the form of a tablet) has a (specified) immediate disintegration and/or an immediate release effect on the release of active substance.

1. A solid pharmaceutical formulation or composition comprising a selectively moisture-conditioned disintegrant.

2. A solid pharmaceutical formulation or composition comprising:
one or more active substances,
one or more selectively moisture-conditioned disintegrants,
and optionally one or more other adjuvants.

3. The solid pharmaceutical formulation or composition according to claim 2, wherein a disintegrant which is not a selectively moisture-conditioned disintegrant, has been replaced by a selectively moisture-conditioned disintegrant.

4. The solid pharmaceutical formulation or composition according to claim 1 in the form of a tablet, wherein the one or more selectively moisture-conditioned disintegrants, the one or more active substances, and optional other adjuvants, if present, are each conditioned to the same specific relative humidity, wherein each of the conditioning processes is carried out below a specific critical relative humidity limit for fragmentation and/or disintegration of the tablet.

5. The solid pharmaceutical formulation or composition according to claim 1 in the form of a tablet, wherein the one or more selectively moisture-conditioned disintegrants, the one or more active substances, and optional other adjuvants, if present, are each conditioned to different specific relative humidities, wherein each of the conditioning processes is carried below a specific critical relative humidity limit for fragmentation and/or disintegration of the tablet.

6. The solid pharmaceutical formulation or composition according to claim 1 in the form of a tablet, wherein the one or more selectively moisture-conditioned disintegrants, the one or more active substances, and optional other adjuvants, if present, are each conditioned to different specific relative humidities, wherein:
the conditioning of the disintegrant is carried out above a specific critical relative humidity limit for fragmentation and/or disintegration of the tablet, and
the conditioning of the active substance and optional other adjuvants, if present, is carried out below a specific critical relative humidity limit for fragmentation and/or disintegration of the tablet.

7. The solid pharmaceutical formulation or composition according to claim 1 in the form of a tablet, wherein the one or more selectively moisture-conditioned disintegrants, the one or more active substances, and optional other adjuvants, if present, are each conditioned to different specific relative humidities, wherein
the conditioning of the disintegrant is carried out below a specific critical relative humidity limit for fragmentation and/or disintegration of the tablet, and
the conditioning of the active substance and optional other adjuvants, if present, is carried out above a specific critical relative humidity limit for fragmentation and/or disintegration of the tablet.

8. A method for preparing a solid pharmaceutical formulation or composition, comprising mixing or combining a disintegrant with one or more active substances and/or one or more other adjuvants, wherein the disintegrant is a selectively moisture-conditioned disintegrant.

9. The method according to claim 8, wherein the moisture-conditioned disintegrant is prepared by:
open storage or storage of the disintegrant in a moisture-permeable packaging under specific climatic conditions, or
mixing highly moisture-laden moisture-saturated disintegrants with dry disintegrants.

10. The solid pharmaceutical formulation or composition according to claim 1, wherein the selectively moisture-conditioned disintegrant is a selectively moisture-conditioned cross-linked polyalkylammonium polymer, crospovidone or derivates thereof having different particle sizes, croscarmellose, starches or derivatives thereof, or sodium starch glycolate.

11. The solid pharmaceutical formulation or composition according to claim 1, wherein the water content of the formulation or composition is from 0.1% to 20% w/w.

12. The solid pharmaceutical formulation or composition according to claim 11, wherein the water content for the formulation or composition is from 1.0% to 5% w/w.

13. The solid pharmaceutical formulation or composition according to claim 1 in the form of a tablet, wherein
the tablet is a delayed disintegration tablet, and/or
the tablet is a delayed release tablet with respect to the active substance.

14. The solid pharmaceutical formulation or composition according to claim 1 in the form of a tablet wherein
the tablet is an immediate disintegration tablet, and/or
the tablet is an immediate release tablet with respect to the active substance.
